(19) United States
(12) Patent Application Publication
(10) Pub. No.: US 2017/0231528 A1
(43) Pub. Date: Aug. 17, 2017

(54) METHOD AND SYSTEM FOR CONTINUOUS MONITORING OF A MEDICAL CONDITION IN PATIENTS

(71) Applicant: Smart Monitor Corp, San Jose, CA (US)

(72) Inventor: Anoo Nathan, San Jose, CA (US)

(73) Assignee: Smart Monitor Corp

(21) Appl. No.: 15/431,538

(22) Filed: Feb. 13, 2017

Related U.S. Application Data
(63) Continuation-in-part of application No. 14/207,495, filed on Mar. 12, 2014.

(51) Int. Cl. A61B 5/11; A61B 5/01; A61B 5/0205; G06N 99/00; A61B 5/00

(52) U.S. Cl. CPC A61B 5/112; G06N 99/005; A61B 5/1118; A61B 5/4806; A61B 5/1112; A61B 5/4082; A61B 5/4088; A61B 5/4094; A61B 5/4504; A61B 5/6801; A61B 5/0205; A61B 5/01; A61B 5/7264; A61B 5/74; G06F 19/363

(57) ABSTRACT

The present invention describes methods and systems to enable a concerned party to continuously monitor the progression of a medical condition in one or more patients. The progression of the medical condition is determined by processing sensor data obtained from one or more physiological and/or motion sensors and survey data obtained from the patients. Further, environmental data such as air quality, temperature and humidity may also be used along with the sensor data and the survey data to monitor/track the progression of the medical condition.

### CROSS REFERENCE TO RELATED APPLICATIONS

This application is a continuation in part of and claims the priority benefit of U.S. Ser. No. 14/207,495 filed on Mar. 12, 2014 the complete contents of both applications are incorporated herein by reference.

### TECHNICAL FIELD

The present invention generally relates to the field of healthcare and medical information services, and in particular, the disclosure relates to methods and systems for continuously monitoring and real time tracking a medical condition in patients.

### BACKGROUND

Continuous monitoring of medical conditions is of great significance for the wellbeing of patients, maintaining medical stability and responding to disastrous conditions (such as a stroke, an epileptic event). In case of chronic diseases, it is beneficial to continuously monitor a patient to detect both exacerbation and remission. However, this leads to an increase in the cost of hospitalization and other related expenses. Therefore, there is a growing demand to create solutions that can enable remote monitoring of the patients without putting their life in risk.

In the healthcare industry, a growing demand for technologies that may enable doctors/caregivers to access current medical status of patients is seen. Further, the evaluation of a patient's medical history/trends to determine the progression of a medical condition or an occurrence of an abnormal event is of utmost importance.

In order to continuously monitor the medical condition of a patient and gather medical information, different types of monitoring devices are in use. Typically, these monitoring devices include physiological sensors and activity monitors. Further, these monitoring devices can be used as standalone devices and/or in combination as well. The medical information captured from the one or more such monitoring devices is used to gain meaningful insights pertinent to progression of the medical condition or occurrence of an abnormal event.

Although, there are various products and applications available in the market for remote patient monitoring, these are not designed for continuous, non-invasive monitoring and therefore do not provide longitudinal data necessary for tracking disease progression proactively. Therefore, there is a need for efficient and accurate ways for continuous monitoring of a medical condition in patients. The present invention is directed towards improved method and/or system that can enable effective monitoring of patients.

### SUMMARY

In the light of the above, it is readily apparent that a need exists in the art for an efficient marketplace for a continuous monitoring and real time tracking of patients suffering from chronic conditions. It is therefore a primary object of the invention to provide a marketplace for patients suffering from chronic conditions to improve quality of life of by providing them with continuous monitoring and real time tracking of the progression of the chronic medical condition. The embodiment of the inventions provides answers to key questions such as, an appropriate time to intervene to prevent acute episodes and hospitalizations needed to enhance the quality of life of patients and patient experience as well as track disease progression of a patient.

An embodiment of the present invention describes a method for continuously monitoring a patient to track the progress of a medical condition. The method includes receiving a first set of parameters by a processing unit. The first set of parameters corresponds to at least one of motion, physical and physiological characteristics of the patient. The method further includes receiving a second set of parameters by the processing unit and the second set of parameters corresponds to information provided by at least one of, but not limited to, a mobile communication device, wearable device, smartphone, tablet, personal digital assistant (PDA) and Internet of Things device. The method further includes correlating the first set of parameters with the second set of parameters by the processing unit. Thereby, at least one of the first set of parameters, the second set of parameters and the correlation between the first set of parameters and the second set of parameters determine the progression of the medical condition.

In another embodiment of the invention, a patient care-flow system describes, a care-flow controller comprising of, a device interaction policy; a device behavioral model; a body-worn device configured for capturing any one of, a physical and, or physiological characteristic as a first set of parameters; a processor, a non-transitory storage element coupled to the processor, encoded instructions stored in the non-transitory storage element, wherein the encoded instructions when implemented by the processor, configure the system to, capture at least the first set of physiological and, or motion parameters of the patient from the body-worn device, at least a second set of contextual parameters of the patient from at least one other device, aggregate device behavior of at least the captured first and a second set of parameters using the device interaction policy, construct a composite behavioral profile based on the aggregated device behavior and comparing composite behavioral profile with a reference behavioral profile by the device behavioral model, assess and alert a patient threat to any one of the patient and, or provider by detecting a discrepancy between the composite behavioral profile and the reference behavioral profile above a predefined threshold and provide an automated response to the assessed and alerted patient threat.

The motion characteristics of the patient correspond to at least one of activity related characteristics and sleep related characteristics of the patient. Examples of activity related characteristics of the patient include, but are not limited to, maximum value of acceleration, minimum value of acceleration, time of acceleration, duration of acceleration, frequency of acceleration, gap between two maximum/minimum values of acceleration, rotational velocity, direction of acceleration, orientation, a stride cycle, a left/right step, a stride length, a walking speed, a stride interval, a gait variability, a stride-to-stride interval and a variability of stride length over time.

Going further, sleep related characteristics of the patient are indicative of at least one of the group comprising sleep time, number of times awake, duration of sound sleep, duration of light sleep and awake time. The physiological characteristics of the patient are one or more of group comprising heart rate, pulse rate, respiratory rate and body temperature.

In an embodiment of the present invention, the first set of parameters is captured by a body worn device of the patient and the second set of parameters is provided by at least one of, a mobile communication device, wearable device, smartphone, tablet, personal digital assistant (PDA) and Internet of Things device. The second set of parameters may be indicative of any one of, but not limited to, fatigue, walking/running/movement related behavior, weakness, bladder dysfunction, anxiety/nervousness, severe headache, nausea, gastrointestinal discomfort, vision problems and speech impairment of the patient.

The method and system further includes generating reports based on at least one of, the first set of parameters, the second set of parameters and the correlation between the first set of parameters and the second set of parameters. The reports are sent to a concerned party such as a healthcare provider, a hospital, a health monitoring service, a doctor, a physician, a clinician, a caregiver and a social service.

In another embodiment of the present invention, the method also includes receiving a third set of parameters by the processing unit. The third set of parameters corresponds to environmental data, and wherein the environmental data includes at least one of, but not limited to, temperature, humidity, air quality, pollen count, carbon dioxide levels and weather data. The method further includes correlating the third set of parameters with the first set of parameters by the processing unit.

Another embodiment of the present invention describes a body worn device. The body worn device includes a processor, a non-transitory storage element coupled to the processor and encoded instructions stored in the non-transitory storage element. The body worn device is configured to capture a first set of parameters of a patient, wherein the first set of parameters corresponds to at least one of motion characteristics and physiological characteristics of the patient. The body worn device is further configured to capture a second set of parameters, wherein the second set of parameters corresponds to information provided by the patient in response to a periodic survey. The first set of parameters and the second set of parameters are then sent to a processing unit using a transceiver.

In an embodiment of the present invention, the body worn device further includes at least one sensor comprising of, a motion sensor, an accelerometer, a 3D accelerometer, a gyroscope, a global positioning system sensor (GPS), a magnetometer, an inclinometer, an impact sensor, a heart rate monitor, a pulse rate monitor, a respiratory rate monitor and body temperature sensor. The body worn device also includes an input unit that enables the patient to provide the information in response to the periodic survey.

### BRIEF DESCRIPTION OF DRAWINGS

FIG. 1 illustrates an exemplary environment in which various embodiments of the disclosure can be practiced.

FIG. 2 illustrates an exemplary body worn device being used by a patient, according an embodiment of the disclosure.

FIG. 3 illustrates an exemplary mobile communication device used for presenting a periodic survey to the patient, according an embodiment of the disclosure.

FIG. 4 illustrates an exemplary processing unit used for monitoring the patient for a medical condition, according an embodiment of the disclosure.

FIG. 5 illustrates an exemplary display screen used for displaying reports on the patient to a concerned party, according an embodiment of the disclosure.

FIG. 6 illustrates a method flowchart for continuously monitoring the patient for the medical condition, according an embodiment of the disclosure.

FIG. 7 is a method flowchart for a method flowchart for continuously monitoring the patient for the medical condition and generating reports, according an embodiment of the disclosure.

FIG. 8 illustrates a process flow of an embodiment of the invention.

FIG. 9 illustrates a process flow of the care-flow controller system of the invention.

FIG. 10 shows an interaction work-flow of the care-flow controller system.

FIG. 11A illustrates an exemplary workflow automation tool embodiment of the invention.

FIG. 11B shows a workflow automation tool embodiment of the invention.

### DETAILED DESCRIPTION OF DRAWINGS

The present invention will now be described more fully with reference to the accompanying drawings, in which embodiments of the invention are shown. However, this disclosure should not be construed as limited to the embodiments set forth herein. Rather, these embodiments are provided so that this disclosure will be thorough and complete, and will fully convey the scope of the disclosure to those skilled in the art. Like numbers refer to like elements throughout.

### Overview

The primary purpose of the disclosure is to enable a concerned party to continuously monitor the progression of a medical condition in one or more patients. Typically, the progression of the medical condition is determined by processing sensor data obtained from one or more physiological and/or motion sensors and survey data obtained from the patients. Further, environmental data such as air quality, temperature and humidity may also be used along with the sensor data and the survey data to monitor/track the progression of the medical condition.

The present disclosure focuses on continuously monitoring the progression of a medical condition in a patient by processing sensor data, survey data and the environmental data. However, for a person skilled in the art it is understood these examples are just for understanding purposes and the disclosure can be implemented for objects other than medical condition monitoring for example fitness level monitoring.

Examples of the medical condition include, but are not limited to, Multiple Sclerosis (MS), Primary Progressive Multiple Sclerosis (PPMS), Huntington's disease (Chorea), Epilepsy & Seizures, Parkinson's disease, Post Stroke conditions, Tobacco use related conditions, Asthma, Cancer, Arthritis, Chronic Obstructive Pulmonary Disease (COPD), Diabetes, heart disease, Obesity, Osteoporosis, Alzheimer's disease, Reflex Sympathetic Dystrophy (RSD) Syndrome, Pruritus and Chronic kidney disease (CKD). Going forward in this disclosure, the present invention will be described taking the example of Multiple Sclerosis. However, for a person skilled in the art it is understood that this example is just for understanding purposes and the disclosure can be implemented for other medical conditions that may result in impairment with respect to motion in the patients.

### Exemplary Environment

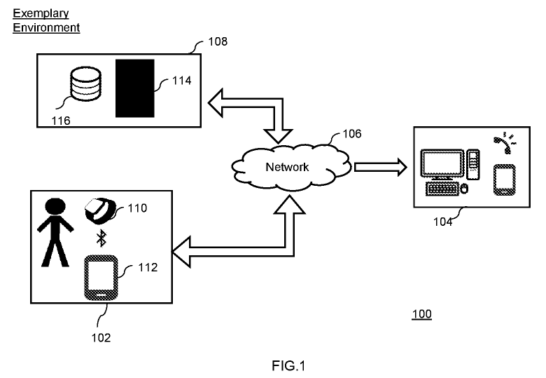

FIG. 1 illustrates an exemplary environment 100 in which various embodiments of the present invention can be practiced. The environment 100 includes patient premises 102, a concerned party 104 and a processing unit 108. The patient premises 102, the concerned party 104 and the processing unit 108 are communicatively coupled through a network 106. Typically, the processing unit 108 enables the concerned party 104 to continuously monitor the symptoms of a medical condition such as Multiple Sclerosis (MS) in the patient, located at the patient premises 102.

Examples of the concerned party 104 include, but are not limited to, a healthcare provider, a hospital, a health monitoring service, a doctor, a physician, a clinician, a caregiver and a social service. The processing unit 108 may either be operated by the concerned party 104 or a third-party. Examples of the third party include, but are not limited to, a service provider that specializes in continuously collecting medical data from patients and distributing the medical data to a plurality of concerned parties. Typically, the processing unit 108 includes a processor 114 and a medical information database 116.

The patient premises 102 is a place, the patient is located at, other than a hospital or any other similar medical institution/setting. To enable continuous monitoring, the patient uses a body worn device 110 and a mobile communication device 112. The body worn device 102 is typically embedded/equipped with one or more motion sensors, physiological sensors and environmental sensors. Examples of these sensors include, but are not limited to accelerometers, gyroscopes, inclinometers, geomagnetic sensors, global positioning systems, impact sensors, microphones, cameras, heart rate monitors, pulse oximeters, blood alcohol monitors, respiratory rate sensors, transdermal sensors, galvanic skin response (GSR) sensors and electromyography (EMG) sensors. In an embodiment of the present invention, the data captured by the one or more sensors is sent to the processing unit 108 through the network 106.

Typically, the body worn device 102 is worn on one or more body parts of the patient, such as wrist, waist, neck, arm, leg, abdomen, chest, thigh, head, ear and fingers. Further, the body worn device 102 may be a wristband, a watch, an armband, a necklace, a headband, an earring, a waist belt and a ring.

The mobile communication device 114 is a portable device that has the capability of communicating over the network 106, presenting periodic surveys to the patient and receiving response from the patient on the periodic surveys. Examples of the mobile communication device 114 include, but are not limited to, a smartphone, a tablet, a personal digital assistant (PDA) and a mobile phone.

In an embodiment of the present invention, the data captured by the one or more sensors of the body worn device 110 is first sent to the mobile communication device 112 and thereby, sent to the processing unit 108 over the network 106. The body worn device 110 communicates with the mobile communication device 112 over a short range wireless communication medium. Examples of the short range wireless communication medium include Bluetooth, ZigBee, Infrared, Near Field Communication (NFC) and Radio-frequency identification (RFID).

The network 106 may be any suitable wired network, wireless network, a combination of these or any other conventional network, without limiting the scope of the present invention. Few examples may include a LAN or wireless LAN connection, an Internet connection, a point-to-point connection, or other network connection and combinations thereof. The network 106 may be any other type of network that is capable of transmitting or receiving data to/from host computers, personal devices, telephones, video/image capturing devices, video/image servers, or any other electronic devices. Further, the network 106 is capable of transmitting/sending data between the mentioned devices. Additionally, the network 106 may be a local, regional, or global communication network, for example, an enterprise telecommunication network, the Internet, a global mobile communication network, or any combination of similar networks. The network 106 may be a combination of an enterprise network (or the Internet) and a cellular network, in which case, suitable systems and methods are employed to seamlessly communicate between the two networks. In such cases, a mobile switching gateway may be utilized to communicate with a computer network gateway to pass data between the two networks. The network 106 may include any software, hardware, or computer applications that can provide a medium to exchange signals or data in any of the formats known in the art, related art, or developed later.

In an embodiment of the present invention, the processing unit 108 receives the sensor data from the body worn device 110 and response on the periodic surveys from the patient. Thereby, the processing unit 108 correlates the sensor data with the response on periodic surveys, generates reports corresponding to the symptoms of medical condition in the patient and sends the reports and other relevant data to the concerned party 104. These reports enable the concerned party 104 to track/monitor the progression of the medical condition in the patient.

In an embodiment of the present invention, the concerned party 104 is enabled to view the reports, as generated by the processing unit 108 using one or more devices selected from the group comprising a smartphone, a computer, a laptop, a tablet, a personal digital assistant (PDA) and a mobile phone.

### Body Worn Device

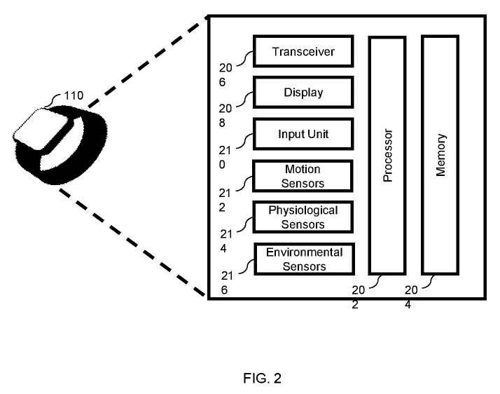

FIG. 2 illustrates the exemplary body worn device 110 being used by the patient, according an embodiment of the disclosure. The body worn device 110 includes a processor 202, a memory 204, a transceiver 206, a display 208 and an input unit 210. The processor 202 executes a set of computer executable instructions stored in the memory 204 for providing the overall functionality of the body worn device 110. The processor 202 is further communicatively coupled to motion sensors 212, physiological sensors 214 and environmental sensors 216.

Continuing with the example of Multiple Sclerosis (MS), it has been observed that walking impairment, fatigue and weakness are the most visible symptoms of MS and affect more than 80% of MS patients. Therefore, these motion related characteristics of the patient are markers of both disability and the progression of MS. In an embodiment of the present invention, the motion sensors 212 and the physiological sensors 212 continuously capture a first set of parameters that reflect motion characteristics and physiological characteristics of the patient respectively.

The motion characteristics of the patient include both activity related characteristics and sleep related characteristics of the patient. Examples of parameters of the first set of parameters, captured by the motion sensors 212, corresponding to activity related characteristics of the patient include, but are not limited to, maximum value of acceleration, minimum value of acceleration, time of acceleration, duration of acceleration, frequency of acceleration, gap between two maximum/minimum values of acceleration, rotational velocity, direction of acceleration, orientation, a stride cycle, a left/right step, a stride length, a walking speed, a stride interval, a gait variability, a stride-to-stride interval and a variability of stride length over time.

In an embodiment of the present invention, parameters of the first set of parameters, captured by the motion sensors 212, corresponding to the sleep related characteristics of the patient are indicative of at least one of the group comprising sleep time/duration, number of times awake, duration of sound sleep, duration of light sleep and awake time.

Examples of the motion sensors 212 include, but are not limited to, accelerometers, 3D accelerometers, gyroscopes, global positioning system (GPS), magnetometers, inclinometers and impact sensors.

Further, examples of parameters of the first set of parameters, captured by the physiological sensors 214 include, but are not limited to, heart rate, pulse rate, respiratory rate and body temperature of the patient.

The environmental sensors 216 of the body worn device 110 capture a third set of parameters corresponding to the environmental data for the current location of the patient. Examples of a parameter of the third set of parameters include, but are not limited to, temperature, humidity, air quality, pollen count, carbon dioxide levels and weather data.

In an embodiment of the present invention, the first set of parameters, as captured by the motion sensors 212 and the physiological sensors 214, are sent to the processing unit 108 over the network 106 by the transceiver 206. In another embodiment of the present invention, the third set of parameters, along with the first set of parameters, are sent to the processing unit 108 by the transceiver 206 over the network 106.

Going further, the display 208 is configured to display reminders and periodic surveys/questionnaires to the patient. The reminders may be medication reminders or reminders for completing a pending periodic survey. In an embodiment of the present invention, the patient is enabled to report his/her symptoms and also, respond to the reminders and the periodic surveys using the input unit 210. In an example, the input unit 210 is a set of one or more push buttons.

### Mobile Communication Device

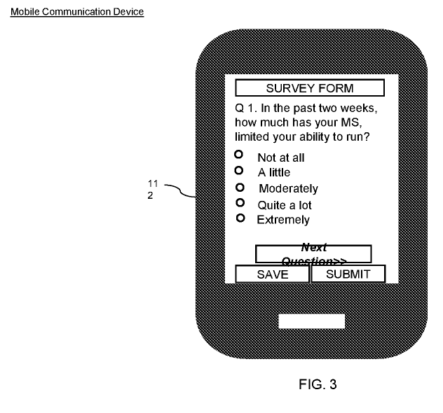

FIG. 3 illustrates the exemplary mobile communication device 112 used for presenting the periodic surveys to the patient, according an embodiment of the disclosure.

The mobile communication device 112 is configured to present periodic surveys to the patient. The response of the patient to questions of a periodic survey determines a second set of parameters. In an embodiment of the present invention, the second set of parameters enable the concerned party 104 to understand patient's perception of changes in his/her motion characteristics such as fatigue, walking/running/movement impairment and weakness. In another embodiment of the present invention, along with the motion characteristics, the second set of parameters are also indicative of at least one of the group comprising bladder dysfunction, vision problems and speech impairment of the patient.

In an example, the periodic survey is a 12 Steps MS Walking Scale (MSWS-12) test. The response of the patient to the test is captured as ordinal measures and thus, generates the second set of parameters.

In an embodiment of the present invention, the mobile communication device 112 is configured to send the second set of parameters to the processing unit 104 using the network 106. As described above, the second set of parameters is determined by the response of the patient to the questions of the periodic survey.

In an embodiment of the present invention, the mobile communication device 112 is also configured to provide the third set of parameters to the processing unit 104.

### Processing Unit

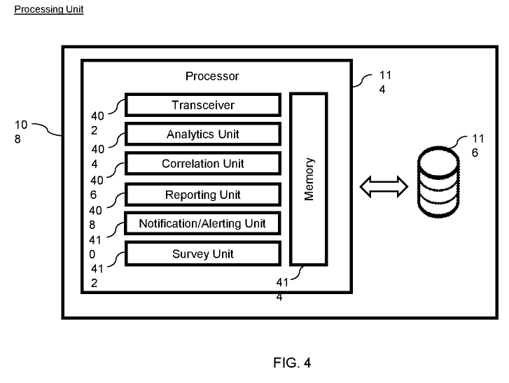

FIG. 4 illustrates the exemplary processing unit 108 used for monitoring the patient for the medical condition, according an embodiment of the disclosure. As shown, the processing unit 108 includes the processor 114 and the medical information database 116. The processor 114 is communicatively coupled to a transceiver 402, an analytics unit 404 and a correlation unit 406. The processor 114 is also communicatively coupled to the medical information database 116 and a memory 414.

The transceiver 402 is configured to receive the first set of parameters from the body worn device 110. Also, the transceiver is further configured to receive the second set of parameters from at least one of the mobile communication device 112 and the body worn device 110. In an embodiment, the transceiver 402 is also configured to receive the third set of parameters from at least one of the body worn device 110 and the mobile communication device 112.

Going further, the analytics unit 404 is configured to analyze the first set of parameters to compute and derive one or more metrics. The one or more metrics are computed using one or more analytical algorithms that are applied on first set of parameters, received continuously from the body worn device 110. In an example, the analytics unit 404 computes metrics corresponding to sustained activity (such as maximum/minimum number of steps in a continuous interval of time), peak activity (such as maximum mean step rate in a 30 minutes' time interval) and walking bouts (such as number of walking bouts, mean duration of walking bouts, mean number of steps in walking bouts and mean cadence of walking bouts) for the patient. However, for a person skilled in the art it is understood that these examples are just for understanding purposes and the disclosure can be implemented for other metrics that may be computed based on the first set of parameters.

In an embodiment of the present invention, the analytics unit 404 is also configured to establish a personal motion signature of the patient based on the first set of parameters. The personal motion signature reflects the normal activity and sleep related characteristics of the patient.

The correlation unit 406 is configured to correlate the second of parameters, as received from the patient in response to the periodic surveys, with at least one of the first set of parameters and the metrics, as computed by the analytics unit 404. In an example, the correlation unit 406 compares the second set of parameters corresponding to the 12 Steps MS Walking Scale (MSWS-12) test with the first set of parameters corresponding to the motion characteristics and physiological characteristics of the patient over a period of 12 months. This enables the concerned party 104 to monitor the activity related characteristics and sleep related characteristics of the patient and compare them with patient's perception of changes in his/her motion characteristics such as fatigue, walking/running/movement impairment and weakness.

In an embodiment of the present invention, the correlation unit 406 is also configured to correlate the first set of parameters with the third set of parameters. This enables the concerned party 104 to monitor the activity related characteristics and sleep related characteristics of the patient and track how these characteristics change with changes in the environmental data.

In an embodiment of the present invention, the processor 114 also includes a reporting unit 408, a notification/alerting unit 410 and a survey unit 412. The reporting unit 408 is configured to generate reports for the patient and provide these reports to the concerned party 104 through the transceiver 402. The reports may be provided to the concerned party at regular intervals (such as daily at 12:00 PM, weekly and monthly), on-demand (when the concerned party 104 requests for a report corresponding to the patient), or when triggered by a condition. Typically, the condition may be defined by the concerned party 104. An example of the condition is when the first set of parameters indicates that the patient has not been active for a predefined interval of time.

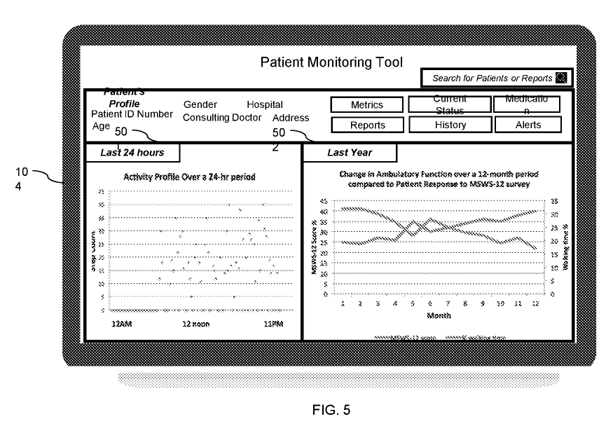

In an embodiment of the present invention, a report, as generated by the reporting unit 408, includes data corresponding to at least one of the group comprising the first set of parameters, the second set of parameters, the third set of parameters and the correlation between the first set of parameters and at least one of the second set of parameters and the third set of parameters. The data shown in the report may be dynamically defined/selected by the concerned party 104. An example of the report, as generated by the reporting unit 408 is shown in FIG. 5. This report is displayed at a computer terminal being used by the concerned party 104 and includes two graphs 501 and 502 for the patient. The graph 501 represents the activity related characteristics i.e. step count of the patient as detected in the last 24 hours. The graph 502 reflects the correlation between the 12-point MSWS test scores over a 12-month period with an activity related characteristic i.e. percentage of walking time for the patient. An increase in MSWS scores indicates a decrease in activity as reported by the patient. This correlates with the decrease in time spent walking over the same 12-month period. The report also includes the profile of the patient including information such as patient identification number, age, gender, address, consulting doctor and hospital. In an embodiment of the present invention, the report also enables the concerned party 104 to view the medical history of the patient, the one or more metrics as computed by the analytics unit 404 for the patient, the current status (real-time) of the patient in terms of the first set of parameters and the second set of parameters, the medication the patient is taking, medical history of the patient, alerts/notifications corresponding to the patient and generate more reports for the patient. Further, the concerned party 104 is also enabled to search for other patients using the patient monitoring tool.

The Notification/Alerting unit 410 is configured to generate reminders for the patient. The reminders may be medication reminders or reminders for completing a pending periodic survey. In an embodiment of the present invention, the reminders are displayed to the patient using the body worn device 110.

In an embodiment of the present invention, the Notification/Alerting unit 410 is also configured to send a notification to the patient and/or the concerned party 104 on detecting a deviation in the first set of parameters with respect to the personal motion signature of the patient using machine learning algorithms. The notification may be a message, a phone call or any other communication means to instantly make the patient and/or the concerned party 104 aware of the deviation.

The survey unit 412 is configured to generate the periodic surveys for the patient and send them to the patient using the transceiver 402. The survey unit 412 may also be configured to determine the questions to be included in the periodic surveys and the frequency at which the periodic surveys are presented to the patient. In an embodiment, the concerned party 104 may configure the survey unit 412 by defining the questions to be included in the periodic surveys and the frequency of presenting these periodic surveys to the patient.

In an embodiment of the present invention, the processor 114 stores the first set of parameters, the second set of parameters and the third set of parameters, as received by the transceiver 402, in the medical information database 116. The medical information database 116 is also used to store the profile of the patient including information such as patient ID number, age, gender, address, medical condition, medical history, medication, reports, the one or more metrics (as computed by the analytics unit 404), questions corresponding to the periodic surveys, frequency of presenting the periodic surveys to the patient, reports (as generated by the reporting unit 408) and the reminders/alerts.

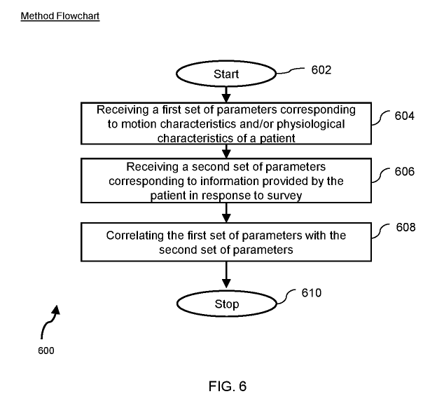

FIG. 6 illustrates a method flowchart for continuously monitoring the patient for the medical condition, according an embodiment of the disclosure. The method 600 starts at step 602.

At step 602, the processing unit 108 receives the first set of parameters from the body worn device 110. The motion sensors 212 and the physiological sensors 212 continuously capture the first set of parameters that reflect motion characteristics and physiological characteristics of the patient respectively. The motion characteristics of the patient include both activity related characteristics and sleep related characteristics of the patient.

At step 604, the processing unit 104 receives the second set of parameters from the mobile communication device 112. The response of the patient to the questions of a periodic survey, as presented by the mobile communication device, determines the second set of parameters. In an embodiment of the present invention, the second set of parameters enable the concerned party 104 to understand the patient's perception of changes in his/her motion characteristics such as fatigue, walking/running/movement impairment and weakness. In another embodiment of the present invention, along with the motion characteristics, the second set of parameters are also indicative of at least one of the group comprising bladder dysfunction, vision problems and speech impairment of the patient.

At step 606, the processing unit 108 correlates the first set of parameters with at least one of the first set of parameters and the one or more metrics, as computed by the analytics unit 404. In an example, the processing unit 108 compares the second set of parameters corresponding to the 12 Steps MS Walking Scale (MSWS-12) test with the first set of parameters corresponding to the motion characteristics and physiological characteristics of the patient over a period of 12 months. This enables the concerned party 104 to monitor the activity related characteristics and sleep related characteristics of the patient and compare them with the patient's perception of changes in his/her motion characteristics such as fatigue, walking/running/movement impairment and weakness.

The method 600 stops at step 608.

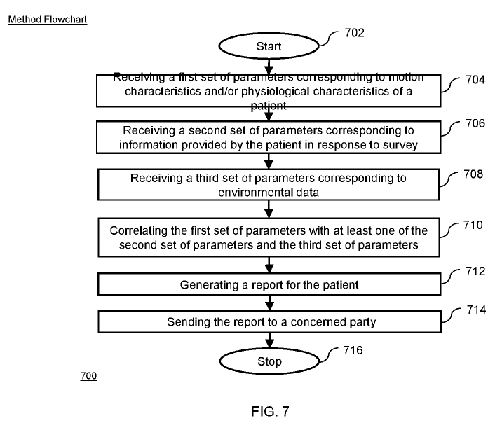

FIG. 7 is a method flowchart for a method flowchart for continuously monitoring the patient for the medical condition and generating reports, according an embodiment of the disclosure. The method 700 starts at step 702.

At step 704, the transceiver 402 of the processor 114 receives the first set of parameters from the body worn device 110. The motion sensors 212 and the physiological sensors 212 of the body worn device 110 continuously capture the first set of parameters that reflect motion characteristics and physiological characteristics of the patient respectively. The motion characteristics of the patient include both activity related characteristics and sleep related characteristics of the patient.

At step 706, the transceiver 402 receives the second set of parameters from the mobile communication device 112. The response of the patient to the questions of a periodic survey, as presented by the mobile communication device, determines the second set of parameters. In an embodiment of the present invention, the second set of parameters enable the concerned party 104 to understand the patient's perception of changes in his/her motion characteristics such as fatigue, walking/running/movement impairment and weakness. In another embodiment of the present invention, along with the motion characteristics, the second set of parameters are also indicative of at least one of the group comprising bladder dysfunction, vision problems and speech impairment of the patient.

At step 708, the transceiver 402 receives the third set of parameters from at least one of the mobile communication device 112. In an embodiment of the present invention, the transceiver 402 receives the third set of parameters from the body worn device 110. The third set of parameters corresponds to the environmental data for the current location of the patient. Examples of a parameter of the third set of parameters include, but are not limited to, temperature, humidity, air quality, pollen count, carbon dioxide levels and weather data.

At step 710, the correlation unit 406 of the processing unit 108 correlates the first set of parameters with at least one of the first set of parameters and the metrics, as computed by the analytics unit 404. In an example, the correlation unit 406 compares the second set of parameters corresponding to the 12 Steps MS Walking Scale (MSWS-12) test with the first set of parameters corresponding to the motion characteristics and physiological characteristics of the patient over a period of 12 months. This enables the concerned party 104 to monitor the activity related characteristics and sleep related characteristics of the patient and compare them with the patient's perception of changes in his/her motion characteristics such as fatigue, walking/running/movement impairment and weakness. In an embodiment of the present invention, the correlation unit 402 correlates the first set of parameters with the third set of parameters. This enables the concerned party 104 to monitor the activity related characteristics and sleep related characteristics of the patient and track how these characteristics change with changes in the environmental data.

At step 712, the reporting unit 408 generates a report for the patient. Typically, the report includes data corresponding to at least one of the group comprising the first set of parameters, the second set of parameters, the third set of parameters and the correlation between the first set of parameters and at least one of the second set of parameters and the third set of parameters. The data shown in the report may be dynamically defined/selected by the concerned party 104.

At step 714, the transceiver 402 sends the report to the concerned party 104 through the network 106. The report may be sent to the concerned party 104 at regular intervals (such as daily at 12:00 PM, weekly and monthly), on-demand (when the concerned party 104 requests for a report corresponding to the patient), or when triggered by a condition. Typically, the condition may be defined by the concerned party 104. An example of the condition is when the first set of parameters indicates that the patient has not been active for a predefined interval of time.

The method 700 stops at step 716.

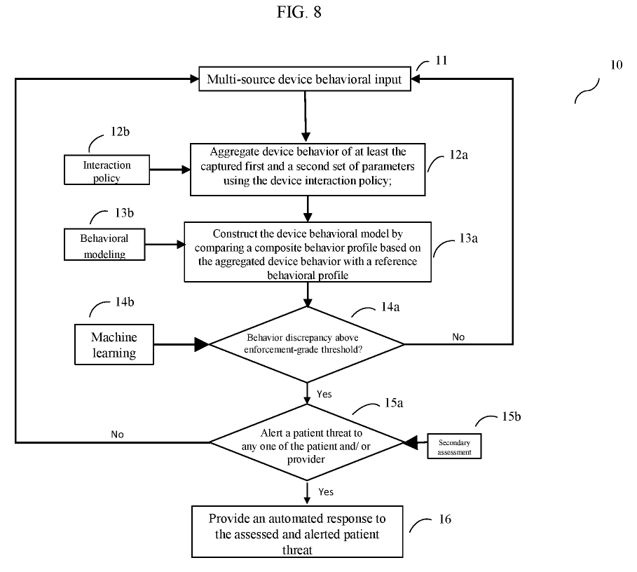

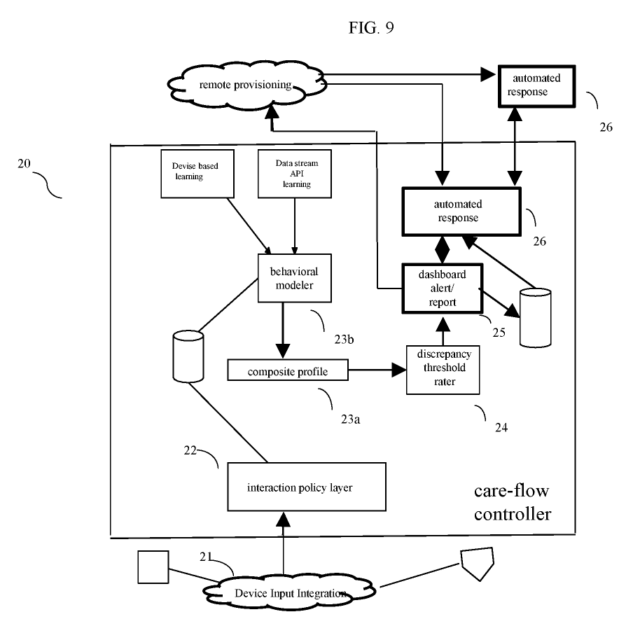

Now in reference to FIGS. 8 and 9—an exemplary process/interaction flow for an integrated patient care-flow system and, or platform for receiving, recognizing, analyzing, and alerting a patient threat based on multiple device inputs is disclosed. The disclosed patient care-flow system and, or platform 10, 20 comprises: a care-flow controller further comprising: a device interaction policy 12b, 22; and a device behavioral model 13b, 23b. The care-flow system and, or platform 10 further comprises a body-worn device configured for capturing any one of a motion characteristic and, or physiological characteristic as a first set of parameters; a processor: a non-transitory storage element coupled to the processor; encoded instructions stored in the non-transitory storage element.

The encoded instructions when implemented by the processor, configure the care-flow system and, or platform 10, 20 to: capture at least the first set of physiological and, or motion parameters of the patient from the body-worn device 11, 21; at least a second set of contextual parameters of the patient from at least one other device 11, 21. The care-flow system and, or platform 10, 20 is further configured to aggregate device behavior of at least the captured first and a second set of parameters 12a, using the device interaction policy 12b, 22.

Once the device input 11, 21—however disparate—is aggregated, the patient care-flow system and, or platform 10, 20 constructs the composite behavior profile 13a, 23a by a behavioral model 13b, 23b and compares said composite behavior profile based on the aggregated device behavior with a reference behavioral profile. If a discrepancy between the composite behavioral profile and the reference behavioral profile is above a pre-defined threshold 14a, then the care-flow system and, or platform 10, 20 assesses the potential threat and alerts the patient of said threat 15a, 25. In addition to alerting the patient, the care-flow system and, or platform 10, 20 may be configured to provide at least one automated response to the assessed and alerted patient threat 16, 26.

In a preferred embodiment, device data input 11, 21 may encompass the sensor-captured raw data input or transduced and processed data input from the body worn device that is the subject of the supra device and method claims. Device input 11, 21 may also encompass the sensor-captured raw data input or transduced and processed data input from any other device associated with the patient/user. Examples may be devices worn, mobile devices, and, or fixed-access devices, such as Internet-of-Things devices (e.g. smart thermostat, home automation consoles, etc.). The plurality of device inputs provides additional input for aggregation and behavior profiling, thus layering the behavior profile with additional context for generating a higher fidelity of predictive analytics. Alternatively, the data input 11, 21 may be from at least two disparate devices—capturing and processing non-overlapping motion or behavior parameters. In yet other embodiments, the data input 11, 21 may be from at least two disparate devices—capturing and processing overlapping motion or behavior parameters. For instance, the system 10, 20 may employ a 6-axis accelerometer data input 11, 21 of the body worn device and the 3-axis accelerometer data input 11, 21 of an additionally worn fitness tracker and stack these motion metrics to generate a composite gait profile. Conversely, the data input 11, 21 may be from at least two disparate devices capturing non-overlapping metrics, such as accelerometer data from the body worn device and gyroscopic data from a fitness tracker to inform the composite gait profile. Even further, these non-overlapping metrics may inform distinct sub-profiles of the composite behavioral profile (gait/posture).

In continuing reference to FIGS. 8 and 9, these multiple and heterogenous data inputs may converge in an integration layer 21—within or external to the care-flow controller. The integration layer may further manage the data packets—of varying format—and collate into discrete bundles of packets/formats. In other embodiments, the integration layer 21 may serve as a data format converter, converting the plurality of data formats—from disparate devices—into a universally recognized format. In yet other embodiments, the plurality of data inputs and formats converge into the interaction policy layer 22 for any one of collating the disparate data formats from a multitude of devices; and, or, converting the disparate data formats into a universally recognized data format; and aggregating the bundled and, or converted data inputs for configuring a composite behavioral profile 13a, 23a.

Devices—however disparate—including the body worn device, can communicate with either integration layer 21 and, or the interaction policy layer 12b, 22 wirelessly via Bluetooth—or any other short-range communication protocol—interfacing with any one of a mobile phone, Wi-Fi router and Wide Area Network access. The care-flow controller aggregates a first set of parameters from the body worn device and a second set of parameters from at least one of, a mobile communication device, wearable device, smartphone, tablet, personal digital assistant (PDA) and Internet of Things device.

The behavioral profiles, both composite and reference, take into account complete device behavior. Device behavior includes not only data output informed by patient/user behavior, but also data output informed by network and device technical characteristics. Such technical characteristics may take into account network traffic, bandwidth, network bottlenecks, network malfunctioning, device malfunctioning, sensor data acquisition fidelity, signal transduction, latency, patient feedback, etc. By taking in such device and network technical characteristics, the system or controller may be able to make a secondary assessment 15b of a discrepancy threshold and rule out device or network malfunctioning—verifying that the threshold discrepancy is due to a patient threat. In some embodiments, the alert of a patient threat and, or automated response is triggered only after the device and, or network anomaly is ruled out after the discrepancy threshold is reached.

Still in reference to FIGS. 8 and 9, the care-flow controller may flag or tag a threshold discrepancy of an event between the composite behavioral profile and the reference behavioral profile 14a, 24 to detect a threat, whereby the threshold discrepancy is determined by machine learning algorithms 14b. Machine learning algorithms 14b may be employed to inform a threshold discrepancy rater 24 to determine whether a discrepancy threshold has been reached. Further yet, a machine learning algorithm may be employed to inform upstream processes: generating a composite behavioral profile and, or the reference behavioral profile 13a, 23a by a behavioral model/er 13b, 23b.

As shown in FIG. 9, embodiments may include the addition of a remote server or cloud server to further provide for back-end functionality and support. The server may be situated adjacent or remotely from the system and connected to each system via a communication network. In one embodiment, the server may be used to support disparate device interaction; user/device behavior history function; disease state assessment; predictive analytics; network sharing function; patient and, or care provider alert function; disease management and, or symptom mitigation tools; and care-flow automation tools. The remote server may be further configured to provide a user-control system, which authenticates the user and retrieves behavioral data of the user, device, and, or network and applies the data against a specific group of users/patients/devices/networks for more accurate predictive modelling and disease state/patient threat assessment.

The network may be any type of network that is capable of transmitting or receiving data to/from/between user devices: computers, personal devices, telephones or any other electronic devices. Moreover, the network may be any suitable wired network, wireless network, a combination of these or any other conventional network, including any one of, or combination of a LAN or wireless LAN connection, an Internet connection, a point-to-point connection, or other network connection—either local, regional, or global. As such, the network may be further configured with a hub, router, node, and, or gateway to serve as a transit point or bridge to pass data between any of the at least networks. The network may include any software, hardware, or computer applications that implement a communication protocol (wide or short) or facilitate the exchange of data in any of the formats known in any art, at any time. In some embodiments, any one of a hub, router, node, and, or gateway may additionally be configured for the body worn device, receiving wearable and, or IoT data, and such data may be integrated, collated, formatted for behavioral profiling, assessment/alerting a patient threat, and, or any other downstream autonomic response. Such contextualized data may further inform the suggestion tool layer or automation tool layer on suggesting reactive or proactive routines within the care-flow.

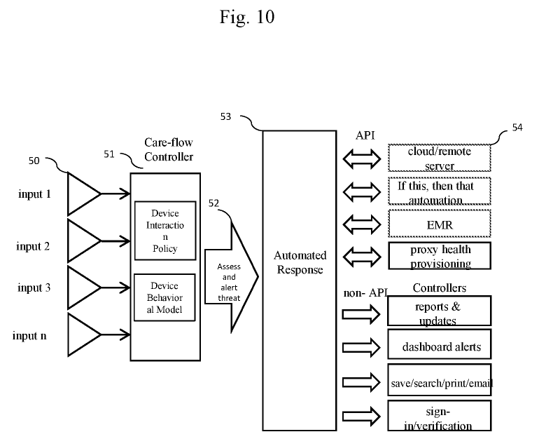

FIG. 10, illustrates an exemplary interaction flow in which various embodiments of the disclosure can be practiced. In a preferred embodiment of the invention, the inputs 50 recognizes a command and processes input from anyone of, multiple sensors on the body worn device, a plurality of devices, patients, caregivers, doctors or concerned party and further, provides the recognized command to the care-flow controller 51. The care-flow controller 51 receives the input 50, recognizes the device behavior via the device interaction policy 51 and is converted and processed for a subsequent assessment of the threat alert 52 for generating a real time automatic response 53 informing the patient, caregiver, doctor or a concerned party of an imminent threat.

Further yet, in an embodiment of the invention, the inputs 50 may be motion characteristics corresponding to at least one of, physical activity, physiological and sleep related characteristics of the patient. Additionally, the physical and physiological activities may have a set of parameters corresponding to activity related characteristics of the patient to be at least one of, but not limited to, maximum/minimum value of acceleration, time of acceleration, duration of acceleration, frequency of acceleration, gap between two maximum/minimum values of acceleration, rotational velocity, direction of acceleration, orientation, a stride cycle, a left/right step, a stride length, a walking speed, a stride interval, a gait variability, a stride-to-stride interval and a variability of stride length over time. Moreover, the sleep related characteristics of the patient may be indicative of at least one of, duration of sleep time, number of times awake, sound sleep, light sleep and awake time. Additionally, environmental conditions may affect patient activity. The environmental conditions can be at least one of, but not limited to, wind velocity, temperature, humidity, aridness, light, darkness, noise pollution, exposure to UV, airborne pollution and radioactivity. Further yet, data generated from a set of parameters corresponding to at least one of, but not limited to, patient reported symptoms and side effects, periodic surveys may be used to generate a behavioral profile of a patient. The data generated may in any one of, but not limited to, audio, video or an image input and further, implemented on at least one of, but not limited to a mobile communication device, body worn device, wearable device, tablet and or IoT.

Additionally, in another embodiment of the invention, the care-flow controller 51, aggregates the data obtained from the body worn device and generates a composite behavioral profile of a patient. Further yet, in another preferred embodiment of the invention, the device behavioral model aggregates a different set of parameters from at least one of, but not limited to, a mobile communication device (e.g. pager), wearable device, smartphone, tablet, personal digital assistant (PDA). Subsequently, after assessing the composite behavioral profile and the reference behavioral profile 52, the care-flow controller may flag a threshold discrepancy of an event between the composite behavioral profile and the reference behavioral profile to detect a threat, whereby the threshold discrepancy is determined by machine learning algorithms.

In another embodiment of the invention, the care-flow controller 51 rules out device and, or network anomaly once a threshold discrepancy is reached by any one of a network traffic analysis, application API interactions, adaptive learning of network/device malfunctioning, and manual feedback of a certain behavior from the patient. Further yet, the alert of a patient threat 52 and, or automated response 53 is triggered only after the device and, or network anomaly is ruled out after the discrepancy threshold is reached. Further yet, in another embodiment of the invention, an automated response 53 in real-time is alerted to at least one of, patient, family member, caregiver or doctors.

Further yet, in another embodiment, the patient care-flow system may further comprise integration with any one of a third-party application via an Application Program Interface (API) 54. This allows for 3rd party database integration, such as Electronic Medical Records (EMR), health monitoring, proxy health provisioning, remote server and, or a cloud based server for other 54 downstream analytics and provisioning. Additionally, the completed automated responses may be saved onto a remote cloud based server for easy access for data acquisition and archival analytics for future use.

In another embodiment of the invention, the care-flow controller 51 may allow for easy saving, searching, printing, and sharing of completed automated response information with authorized participants. Additionally, the care-flow controller may allow for non-API applications, for example, building reports and updates, create dashboard alerts as well as sign in/verifications 54. Alternatively, sharing may be possible with less discrimination based on select privacy filters.

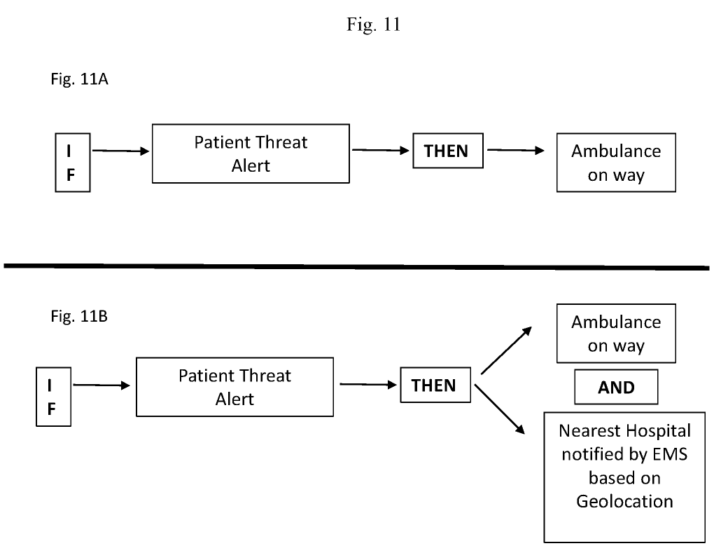

FIG. 11A-B, illustrates a workflow automation tool diagram for prompting the system to perform a task command, provided a trigger is activated based on the threshold discrepancy. In an embodiment of the invention, at least one conditional event triggers at least one action controlled by a “if this, then that” script manager. Further yet, the “if this, then that” script manager is embedded with an “and, or” trigger or action operators, allowing increased triggers or actions in a command set.

In a preferred embodiment of the invention, for instance, for a patient suffering from epileptic seizure, as shown in FIG. 11A, “IF” a threat is alerted to a patient “THEN” the ambulance will be on the way. In another instance, the script manager may be embedded with a “if, this, then that” as well as a “and, or” trigger or action operator for increased triggers either downstream or upstream of a command set. As shown in FIG. 11B, “IF” a threat is alerted to a patient, “THEN”, the ambulance will be alerted “AND” the nearest hospital will be notified about the incoming patient for a transfer of EMR records based on geolocation. All of the commands are automatically triggered once a discrepancy threshold is reached.

In yet another embodiment of the invention, “OR” operators may be used instead of the “AND” operator. Further, any number of “AND” and, or “OR” operator may be used in a command function. Such an automation layer may add further efficiencies to the patient care-flow. This ecosystem of apps may provide for a link to the care-flow controller for enhanced co-interactivity among patient and care providers, diagnostics, and other measurables.

In yet another embodiment of the invention, the body worn device and alert notification mechanism generated by the care-flow system is further configured to, either receive or transmit an automated response of at least one of, notification reminders, notifications, medication reminders, activity confirmations, health analysis, health checks, from at least one of, device, mobile phone, tablet, email, text or internet server. Additionally, the automated response may be anyone of, or a combination of, duration, frequency and severity analytics of threat episodes suffered by a patient. Further yet, in another embodiment, the alert notification is at least one of, text, email, vibration with or without audible notification, visual display, and, or a color-coded or blinking notification.

Although the present disclosure describes the invention with respect to only one patient and concerned party, for a person skilled in the art it is understood that this example is just for understanding purposes and the disclosure can be implemented for monitoring a plurality of patients and a plurality of concerned parties.

In some embodiments, the method flowchart of FIG. 6 and FIG. 7 may be implemented in any suitable hardware, software, firmware, or combination thereof, that exists in the related art or that is later developed.

In the drawings and specification, there have been disclosed exemplary embodiments of the disclosure. Although specific terms are employed, they are used in a generic and descriptive sense only and not for purposes of limitation, the scope of the invention being defined by the following claims. Those skilled in the art will recognize that the present invention admits of a number of modifications, within the spirit and scope of the inventive concepts, and that it may be applied in numerous applications, only some of which have been described herein. It is intended by the following claims to claim all such modifications and variations which fall within the true scope of the invention.

Embodiments described in the present disclosure can be implemented by any system having a processor and a non-transitory storage element coupled to the processor, with encoded instructions stored in the non-transitory storage element. The encoded instructions when implemented by the processor configure the system to continuously monitor the plurality of patients as discussed above in FIGS. 1-11. The systems shown in FIGS. 1-5 and 8-11 can practice all or part of the recited methods (FIGS. 6 and 7), can be a part of the recited systems, and/or can operate according to instructions in the non-transitory storage element. The non-transitory storage element can be accessed by a general purpose or special purpose computer, including the functional design of any special purpose processor. Few examples of such non-transitory storage element can include RAM, ROM, EEPROM, CD-ROM or other optical disk storage or other magnetic. The processor and non-transitory storage element (or memory) are known in the art, thus, any additional functional or structural details are not required for the purpose of the current disclosure.

Embodiments are described at least in part herein with reference to flowchart illustrations and/or block diagrams of methods, systems, and computer program products and data structures according to embodiments of the disclosure. It will be understood that each block of the illustrations, and combinations of blocks, can be implemented by computer program instructions. These computer program instructions may be provided to a processor of a general-purpose computer, special purpose computer, or other programmable data processing apparatus to produce a machine such that the instructions, which execute via the processor of the computer or other programmable data processing apparatus, create means for implementing the functions/acts specified in the block or blocks.

What is claimed is:

1. A method for continuously monitoring a patient to track the progress of a medical condition, the method comprising:
receiving a first set of parameters by a processing unit, wherein the first set of parameters corresponds to at least one of motion characteristics and physiological characteristics of the patient;
receiving a second set of parameters by the processing unit, wherein the second set of parameters corresponds to information provided by the patient in response to a periodic survey; and
correlating the first set of parameters with the second set of parameters by the processing unit;
whereby, at least one of the first set of parameters, the second set of parameters and the correlation between the first set of parameters and the second set of parameters determine the progress of the medical condition.

2. The method of claim 1, further comprising receiving a third set of parameters by the processing unit, wherein the third set of parameters corresponds to environmental data, and wherein the environmental data includes at least one of the group comprising temperature, humidity, air quality, pollen count, carbon dioxide levels and weather data.

3. The method of claim 1, further comprising correlating the third set of parameters with the first set of parameters by the processing unit.

4. The method of claim 1, wherein the medical condition is selected from the group comprising Multiple Sclerosis (MS), Primary Progressive Multiple Sclerosis (PPMS), Huntington's disease, Chorea, Epilepsy, Parkinson's disease, Seizures Post Stroke conditions, Tobacco use related conditions, Asthma, Cancer, Arthritis, Chronic Obstructive Pulmonary Disease (COPD), Diabetes, heart disease, Obesity, Osteoporosis, Alzheimer's disease, Reflex Sympathetic Dystrophy (RSD) Syndrome, Pruritus and Chronic kidney disease (CKD).

5. The method of claim 1, wherein the motion characteristics of the patient correspond to at least one of activity related characteristics and sleep related characteristics of the patient.

6. The method of claim 5, wherein a parameter of the first set of parameters corresponding to the activity related characteristics of the patient is at least one of the group comprising maximum value of acceleration, minimum value of acceleration, time of acceleration, duration of acceleration, frequency of acceleration, gap between two maximum/minimum values of acceleration, rotational velocity, direction of acceleration, orientation, a stride cycle, a left/right step, a stride length, a walking speed, a stride interval, a gait variability, a stride-to-stride interval and a variability of stride length over time.

7. The method of claim 5, wherein a parameter of the first set of parameters corresponding to the sleep related characteristics of the patient is indicative of at least one of the group comprising sleep time, number of times awake, duration of sound sleep, duration of light sleep and awake time.

8. The method of claim 5, wherein the first set of parameters corresponding to the motion characteristics of the patient are captured by one or more sensors selected from the group comprising a motion sensor, an accelerometer, a 3D accelerometer, a gyroscope, a global positioning system sensor (GPS), a magnetometer, an inclinometer and an impact sensor.

9. The method of claim 1, wherein a parameter of the first set of parameters corresponding to physiological characteristics of the patient is one of group comprising heart rate, pulse rate, respiratory rate and body temperature.

10. The method of claim 1, wherein the first set of parameters is captured by a body worn device of the patient.

11. The method of claim 10, wherein the second set of parameters is provided by the patient using at least one of a mobile communication device and the body worn device of the patient.

12. The method of claim 1, wherein a parameter of the second set of parameters is indicative of at least one of the group comprising fatigue, walking/running/movement related impairment, weakness, bladder dysfunction, vision problems and speech impairment of the patient.

13. The method of claim 1, further comprising generating reports based on at least one of the first set of parameters, the second set of parameters and the correlation between the first set of parameters and the second set of parameters.

14. The method of claim 13, further comprising sending the reports to a concerned party, wherein the concerned party is at least one of the group comprising a healthcare provider, a hospital, a health monitoring service, a doctor, a physician, a clinician, a caregiver and a social service.

15. The method of claim 1, further comprising establishing a personal motion signature of the patient based on the first set of parameters.

16. The method of claim 15, further comprising detecting a deviation from the personal motion signature of the patient using machine learning algorithms.

17. The method of claim 16, further comprising sending a notification to at least one of the patient and a concerned party when the deviation from the personal motion signature of the patient is detected.

18. A body worn device comprising:
a processor, a non-transitory storage element coupled to the processor, encoded instructions stored in the non-transitory storage element, wherein the encoded instructions when implemented by the processor, configure the body worn device to:
capture a first set of parameters of a patient, wherein the first set of parameters corresponds to at least one of motion characteristics and physiological characteristics of the patient;
capture a second set of parameters, wherein the second set of parameters corresponds to information provided by the patient in response to a periodic survey; and
sending the first set of parameters and the second set of parameters to a processing unit using a transceiver;
whereby, at least one of the first set of parameters, the second set of parameters and a correlation between the first set of parameters and the second set of parameters determine the progress of a medical condition in the patient.

19. The body worn device of claim 18 further comprising one or more sensors selected from the group comprising a motion sensor, an accelerometer, a 3D accelerometer, a gyroscope, a global positioning system sensor (GPS), a magnetometer, an inclinometer, an impact sensor, a heart rate monitor, a pulse rate monitor, a respiratory rate monitor and body temperature sensor.

20. The body worn device of claim 18 further comprising an input unit, wherein the patient provides the information in response to the periodic survey using the input unit.

21. A patient care-flow system, said system comprising:
a care-flow controller comprising:
a device interaction policy;
a device behavioral model;
a body-worn device configured for capturing any one of a physical and, or physiological characteristic as a first set of parameters;
a processor:
a non-transitory storage element coupled to the processor;
encoded instructions stored in the non-transitory storage element, wherein the encoded instructions when implemented by the processor, configure the system to:
capture at least the first set of physiological and, or motion parameters of the patient from the body-worn device; at least a second set of contextual parameters of the patient from at least one other device;
aggregate device behavior of at least the captured first and a second set of parameters using the device interaction policy;
construct a composite behavioral profile based on the aggregated device behavior and comparing said composite behavioral profile with a reference behavioral profile by the device behavioral model;
assess and alert a patient threat to any one of the patient and, or provider by detecting a discrepancy between the composite behavioral profile and the reference behavioral profile above a predefined threshold; and
provide an automated response to the assessed and alerted patient threat.

22. The patient care-flow system of claim 21, wherein the first set of parameters corresponding to motion and, or physiological characteristics of the patient from the body worn device is at least one of, maximum and minimum value of acceleration, time of acceleration, duration of acceleration, frequency of acceleration, gap between two maximum/minimum values of acceleration, rotational velocity, direction of acceleration, orientation, a stride cycle, a left/right step, a stride length, a walking speed, a stride interval, a gait variability, a stride-to-stride interval and a variability of stride length over time.

23. The patient care-flow system of claim 21, wherein a set of parameters corresponding to an environmental condition surrounding the patient is at least one of, wind velocity, temperature, humidity, aridness, light, darkness, noise pollution, exposure to UV, airborne pollution and radioactivity.

24. The patient care-flow system of claim 21, wherein the care-flow controller aggregates a second set of parameters from at least one of, a mobile communication device, wearable device, smartphone, tablet, personal digital assistant (PDA) and Internet of Things device.

25. The patient care-flow system of claim 21, wherein the care-flow controller may flag a threshold discrepancy of an event between the composite behavioral profile and the reference behavioral profile to detect a threat, whereby the threshold discrepancy is determined by machine learning algorithms.

26. The patient care-flow system of claim 21, wherein the care-flow controller rules out device and, or network anomaly once a threshold discrepancy is reached by any one of a network traffic analysis; application API interactions; adaptive learning of network/device malfunctioning; and manual feedback of a certain behavior from the patient.

27. The patient care-flow system of claim 26, wherein the alert of a patient threat and, or automated response is triggered only after the device and, or network anomaly is ruled out after the discrepancy threshold is reached.

28. The patient care-flow system of claim 21, further comprising integration with any one of a third-party application via an Application Program Interface (API).

29. The patient care-flow system of claim 21, further comprising integration with any one of, electronic medical records (EMR), remote server, and, or a cloud-based server for down-stream analytics and, or provisioning.

30. The patient care-flow system of claim 21, wherein at least one conditional event triggers at least one action controlled by a “if this, then that” script manager.

31. The system of claim 21, wherein a “if this, then that” script manager is further embedded with an “and, or” trigger or action operators, allowing increased triggers or actions in a command set.

32. The patient care-flow system of claim 21, wherein the patient threat alert is at least one of, text, email, vibration with or without audible notification, visual display, and, or a color-coded or blinking notification.

33. The patient care-flow system of claim 21, wherein the automated response is anyone of, or a combination of, duration, frequency and severity analytics of threat episodes.

34. The patient care-flow system of claim 21, wherein the assessed and alerted patient threat is related to any of, or combination of, Multiple Sclerosis (M.S.), Primary Progressive Multiple Sclerosis (PPMS), Huntington's Disease, Epilepsy, Chorea, Parkinson's Disease, Seizures, and, or any post-stroke conditions.
